(19) United States
(12) Patent Application Publication
(10) Pub. No.: US 2025/0182650 A1
(43) Pub. Date: Jun. 5, 2025

(54) MODEL EYE HAVING POSTERIOR CORNEA REFLECTIVITY SIMILAR TO HUMAN EYE

(71) Applicant: Huvitz Co., Ltd., Anyang-si (KR)

(72) Inventors: Ye Na JU, Anyang-si (KR); Sunhyung JO, Anyang-si (KR); Woo Kyung BAEK, Anyang-si (KR)

(21) Appl. No.: 18/964,012

(22) Filed: Nov. 29, 2024

(30) Foreign Application Priority Data
Dec. 4, 2023 (KR) 10-2023-0173411

(51) Int. Cl. G09B 23/30

(52) U.S. Cl. CPC G09B 23/30

(57) ABSTRACT

A model eye having a reflectivity of the posterior cornea similar to that of a human eye is disclosed. The model eye of the present disclosure may include: a body having a space formed therein; solid glass mounted in front of the body to seal the body, as a substitute for the cornea of a human eye; and a liquid substance being vegetable oil filled in the space of the body. The liquid substance may be sunflower oil. The glass may be H-FK95 glass.

### CROSS-REFERENCE TO RELATED APPLICATION

This application claims priority from Korean Patent Application No. 10-2023-0173411 filed on Dec. 4, 2023, which is incorporated herein by reference in its entirety.

### TECHNICAL FIELD

The present disclosure relates to a model eye and, more particularly, to a model eye having a posterior cornea reflectivity similar to that of a human eye.

### RELATED ART

FIG. 1 is a diagram to explain the structure of the anterior part of a typical human eye. As shown in FIG. 1, the anterior part of the human eye is composed of the cornea located in the outermost layer, the iris located in the middle layer of the eye to control the amount of light, and the lens corresponding to the lens of a camera. The anterior chamber, the space between the cornea and the lens, is filled with a clear, water-like liquid.

The average curvature of the anterior cornea is approximately 7.8 mm, and the average curvature of the posterior cornea is approximately 6.5 mm.

Meanwhile, corneal thickness measurement using an optometry instrument (full cornea pachimetry) is performed by calculating the width using signals from the front and back of the cornea that are reflected after projecting a slit beam toward the cornea. In this process, when light passes through different media, the reflection coefficient (p) of the light changes depending on the refractive indexes of the media. When light is incident through medium 1 and reflected vertically from medium 2, the reflection coefficient (p) of light at the boundary between medium 1 and medium 2 may be calculated according to Equation 1 below.

$$ρ = \frac{{n}_{1} - {n}_{2}}{{n}_{1} + {n}_{2}} \qquad [ Equation 1 ]$$

In Equation 1 above, n₁ is the refractive index of the region where light is incident (medium 1), and n₂ is the refractive index of the region where light is reflected (medium 2). The medium in front of the cornea is air with a refractive index (n) of approximately 1, the refractive index of the cornea is 1.3771, and the medium behind the cornea is a liquid with a refractive index of approximately 1.3374.

In the related art, to develop equipment to examine various symptoms or diseases of the cornea, model eyes that can refer to the shape of the cornea have been used, but there is no model eye that has the reflectivity of the anterior and posterior cornea similar to that of a human eye. That is, in the related art, model eyes have been utilized in which glass with a curvature similar to that of the cornea is used as a substitute for the cornea and the back of the cornea i.e. the anterior chamber is filled with water.

Table 1 below shows the refractive indices and reflection coefficients of the anterior cornea and posterior cornea of a human eye and the refractive indices and reflection coefficients of a related-art model eye.

TABLE 1

| | | | | Anterior | Posterior |
|---|---|---|---|---|---|
| | | | Air | cornea | Cornea |
| Human eye | | Refractive | 1 | 1.3771 | 1.3374 |
| | | index (n) | | | |
| | | Reflection | | 0.159 | 0.015 |
| | | coefficient | | | |
| Glass No. 1 | Water | Refractive | 1 | 1.458 | 1.3332 |
| (Fused silica) | | index (n) | | | |
| n = 1.458 | | Reflection | | 0.186 | 0.045 |
| | | coefficient | | | |

As can be seen in Table 1, for the human eye, the refractive index and the reflection coefficient at the anterior cornea are approximately 1.3771 and 0.159, and the refractive index and the reflection coefficient at the posterior cornea are approximately 1.3374 and 0.015. On the other hand, for the related-art model eye, the refractive index of the quartz glass (fused silica) and the reflection coefficient at the front of the quartz glass are approximately 1.458 and 0.186, and the refractive index of the water behind the quartz glass and the reflection coefficient at the back of the quartz glass are 1.3332 and 0.045. There are large deviations from the reflection coefficients of the anterior cornea and posterior cornea, so there is a problem in that good corneal signals cannot be obtained from the related-art model eye.

### PRIOR ART DOCUMENTS

(Patent document 1) Korean Patent Publication No. KR 10-2012-0115807 A
(Patent document 2) Korean Patent Publication No. KR 10-2010-0121927 A
(Patent document 3) Korean Patent Publication No. KR 10-2022-0152655 A

### SUMMARY

An object of the present disclosure is to provide a model eye having a posterior cornea reflectivity similar to that of a human eye.

To achieve the above objectives, the model eye of the present disclosure having a posterior cornea reflectivity similar to that of a human eye may include: a body having a space formed therein; solid glass mounted on the front of the body to seal the body, as a substitute for the cornea of a human eye; and a liquid substance being vegetable oil filled in the space of the body.

The liquid substance may be sunflower oil.

The glass may be H-FK95 glass.

According to the model eye of the present disclosure, the reflection coefficients of the front and back surfaces of the glass of the model eye are similar to the reflection coefficients of the anterior cornea and the posterior cornea of a human eye, so that a higher quality measurement signal can be obtained when developing ophthalmic equipment such as a corneal thickness measurement instrument, and it can also be usefully utilized for the calibration of an optometric instrument.

### BRIEF DESCRIPTION OF THE DRAWINGS

FIG. 1 is a diagram describing the anterior structure of a human eye.

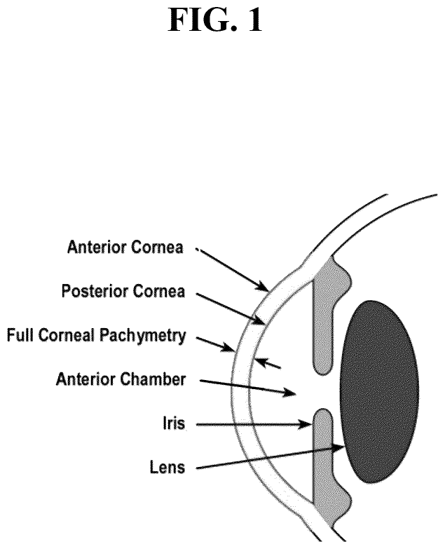

FIG. 2 is a schematic cross-sectional view of a model eye having a posterior cornea reflectivity similar to that of a human eye according to the present disclosure.

### DETAILED DESCRIPTION

Hereinafter, preferred embodiments of the present disclosure will be described in detail with reference to the accompanying drawings.

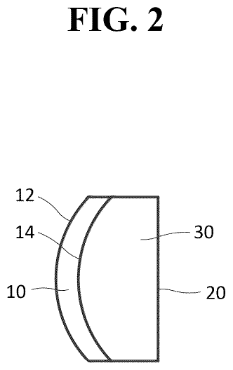

FIG. 2 is a schematic cross-sectional view of a model eye having a posterior cornea reflectivity similar to that of a human eye according to the present disclosure.

As shown in FIG. 2, the model eye of the present disclosure is composed of a body 20 filled with a liquid substance 30, preferably vegetable oil, in a space corresponding to the anterior chamber of a human eye, and solid glass 10 corresponding to the cornea of the human eye and mounted on the front surface of the body 20.

In the above-described configuration, the body 20 may be implemented with a metal material or a plastic material. The glass 10 seals the body 20 to prevent the liquid substance 30 filled in the body 20 from leaking, and its front surface 12 and back surface 14 are formed to have curvatures corresponding to the anterior and posterior corneal curvatures of a human eye.

Table 2 below shows the refractive index of vegetable oils that may be filled in the internal space of the body 20.

TABLE 2

| | |
|---|---|
| Coconut oil | 1.452 |
| Olive oil | 1.466 |
| Canola oil | 1.474 |
| Sunflower oil | 1.475 |

As shown in Table 2, it may be seen that the refractive indices of representative vegetable oils are in the range of approximately 1.45 to 1.48.

Table 3 below is a table comparing the refractive index and reflection coefficient of the model eye of the present disclosure, when various types of glass and sunflower oil filling materials are adopted, with the reflection coefficient of the cornea of a human eye.

TABLE 3

| | | | | Anterior | Posterior |
|---|---|---|---|---|---|
| | | | Air | cornea | cornea |
| Human eye | | Refractive | 1 | 1.3771 | 1.3374 |
| | | index (n) | | | |
| | | Reflection | | 0.159 | 0.015 |
| | | coefficient | | | |
| Glass No. 1 | Water | Refractive | 1 | 1.458 | 1.3332 |
| (Fused silica) | | index (n) | | | |
| n = 1.458 | | Reflection | | 0.186 | 0.045 |
| | | coefficient | | | |
| | Sunflower | Refractive | 1 | 1.458 | 1.475 |
| | oil | index (n) | | | |
| | | Reflection | | 0.186 | 0.006 |
| | | coefficient | | | |
| Glass No. 2 | Water | Refractive | 1 | 1.438 | 1.3332 |
| (H-FK95) | | index (n) | | | |
| n = 1.438 | | Reflection | | 0.180 | 0.038 |
| | | coefficient | | | |
| | Sunflower | Refractive | 1 | 1.438 | 1.475 |
| | oil | index (n) | | | |
| | | Reflection | | 0.180 | 0.013 |
| | | coefficient | | | |
| Glass No. 3 | Water | Refractive | 1 | 1.457 | 1.3332 |
| (H-FK71) | | index (n) | | | |
| n = 1.457 | | Reflection | | 0.186 | 0.044 |
| | | coefficient | | | |
| | Sunflower | Refractive | 1 | 1.457 | 1.475 |
| | oil | index (n) | | | |
| | | Reflection | | 0.186 | 0.006 |
| | | coefficient | | | |

As shown in Table 3, the refractive index of fused silica is 1.458, the refractive index of H-FK95 glass is 1.438, and the refractive index of H-FK71 glass is 1.457, so it can be seen that the refractive index of the glass that can be used in the present disclosure is in the range of approximately 1.4 to 1.5, specifically, 1.43 to 1.46.

Meanwhile, comprehensively considering the reflection coefficients of the anterior cornea and the posterior cornea of a human eye, it can be seen that when glass 10 having a refractive index of 1.43 to 1.45, for example, H-FK95 glass 10 having a refractive index of 1.438, is adopted as a corneal substitute, and a liquid substance 30 having a refractive index of 1.465 to 1.485, for example, sunflower oil having a refractive index of 1.475, is adopted as a filling material 30 of the anterior chamber space, the deviation between the reflection coefficients of the front and back surfaces of the glass of the model eye and the reflection coefficients of the anterior cornea and the posterior cornea of the human eye is minimized.

According to the model eye of the present disclosure, the reflection coefficients of the front and back surfaces of the glass of the model eye are similar to the reflection coefficients of the anterior cornea and the posterior cornea of a human eye, so that a higher quality measurement signal can be obtained when developing ophthalmic equipment such as a corneal thickness measurement instrument, and it can also be usefully utilized for the calibration of an optometric instrument.

Although the embodiments of the present disclosure have been described above, the present disclosure is not limited to those embodiments described above.

The scope of the following claims should be interpreted to encompass all modifications, and equivalent structures and functions of the exemplary embodiments.

### Description of Reference Symbols

10: corneal replacement glass
12: front surface of glass
20: body
30: liquid filling substance

What is claimed is:

1. A model eye having a posterior cornea reflectivity similar to that of a human eye, comprising:
a body having a space formed therein;
solid glass mounted in front of the body to seal the body, as a substitute for a cornea of a human eye; and
a liquid substance being vegetable oil filled in the space of the body.

2. The model eye of claim 1, wherein the liquid substance is sunflower oil.

3. The model eye of claim 1, wherein the glass is H-FK95 glass.

4. The model eye of claim 1, wherein a refractive index of the liquid substance is between 1.465 and 1.485, and a refractive index of the glass is between 1.43 and 1.45.
